(19) United States
(12) Patent Application Publication
(10) Pub. No.: US 2013/0309755 A1
(43) Pub. Date: Nov. 21, 2013

(54) Fungal Leachate and Method of Making Same

(71) Applicants: Gavin McIntyre, Troy, NY (US); Jacob Winiski, Watervliet, NY (US); Sue Van Hook, Cambridge, NY (US); Lucy Greetham, Troy, NY (US); Courtney Hart, Adams, MA (US)

(72) Inventors: Gavin McIntyre, Troy, NY (US); Jacob Winiski, Watervliet, NY (US); Sue Van Hook, Cambridge, NY (US); Lucy Greetham, Troy, NY (US); Courtney Hart, Adams, MA (US)

(21) Appl. No.: 13/875,425

(22) Filed: May 2, 2013

Related U.S. Application Data
(60) Provisional application No. 61/642,491, filed on May 4, 2012.

(51) Int. Cl. C12N 1/14

(52) U.S. Cl. CPC C12N 1/14
USPC 435/256.8

(57) ABSTRACT

A method of producing a fungal leachate solution comprises the steps of obtaining a feedstock of lignocellulosic substrate; colonizing the substrate with a selected fungus; and adding water to the colonized substrate to form a liquid medium containing at least one of sugar alcohol, a phenolic compound and a fatty acid. A leachate from the liquid medium can be used as a liquid culture medium to culture fungi.

This application claims the benefit of U.S. Provisional Patent Application 61/642,491 filed May 5, 2012.

This invention relates to a fungal leachate and to a method of making a fungal leachate.

### BACKGROUND OF THE INVENTION

It is common practice to culture fungi in liquid culture media. Typically, such media are comprised of water, a water-insoluble material, a sugar such as sucrose, and other nutrients. This is well documented in such patents as U.S. Pat. No. 2,693,665 and U.S. Patent Application Pub. No.: US 2003/0070351. These processes typically utilize complex combinations of specifically produced nutrient components, which requires extensive processing and mixing and can be prohibitively costly at scale.

The practice of utilizing waste streams from various substrates and manufacturing processes to produce liquid media for cultivating fungal biomass has been well explored. One characteristic common amongst waste fluid and manufacturing by-product derived fungal growing media is their dependence on economically important crops and processes. U.S. Patent Application Pub. No.: US 2008/0153149 provides a good example describing the use of thin stillage from dry-grind corn milling within ethanol production processes as a growing medium for fungi.

Other liquids and liquid by-products, such as the wash water from rice and wheat production, as well as coconut water have been shown to be viable liquid media for growing fungi as well. None of these examples utilize a fungus as the primary agent for processing lignocellulose into water soluble components that can be used to produce liquid culture media.

A process is also known to produce a spent mushroom bed extract that can be used to cultivate fungi. This process focuses on extraction of enzymes and other bioactive compounds utilizing a complex extraction process dependent on homogenization, centrifugation, and filtration.

Accordingly, it is an object of the invention to employ leachate as a liquid culture medium for cultivating fungi.

It is another object of the invention to provide a fungal leachate that does not require additional nutrient components.

It is another object of the invention to provide a process for producing leachate in a less energy intensive manner than prior art processes.

Briefly, the invention provides a fungal leachate solution containing a water soluble fraction of fungally colonized and decomposed lignocellulosic substrate.

In addition, the invention provides a method of producing a fungal leachate solution comprising the steps of obtaining a feedstock of lignocellulosic substrate; colonizing the substrate with a selected fungus; and adding water to the colonized substrate to form a liquid medium containing at least one of sugar alcohol, a phenolic compound and a fatty acid.

In accordance with the method, a lignocellulose substrate—which has been colonized by a species within Basidiomycetes, Ascomycetes, or Zygomycetes—is leached in water to obtain a combination of water soluble products from the lignocellulose substrate, fungal biomass, and products specifically resulting from the fungal decomposition of the substrate, resulting in a leachate that contains all the nutrition necessary to be utilized as a culture medium for fungi.

This leachate (hereinafter referred to as “leachate” or “fungal leachate”) can be used for the same applications and processes that traditional fungal culture media (such as malt extract agar) are used. This functionality of leachate is specifically dependent on the by-products of fungal decomposition of lignocellulose. Application examples would include cultivation of sheets of mycelium, gelatinized leachate to produce a tissue suspension medium for obtaining 3D colonization through the volume of the fluid, or used as an additive to solid substrate.

In accordance with the invention, the method may be varied to include 1) a process whereby the pre-colonized lignocellulose feedstock both produces the leachate medium and the inoculum (via mycelial fragments and/or asexual spores) in a single process, and 2) leachate derived from rye straw (colonized and decomposed as described above) which demonstrates a specific effectiveness at stimulating germination of asexual spores, namely chlamydospores.

These and other objects and advantages of the invention will become more apparent from the following detailed description taken in conjunction with the accompanying drawings wherein:

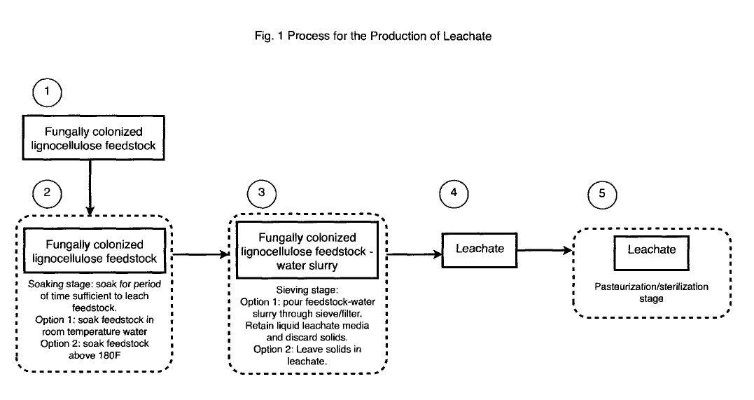

FIG. 1 illustrates a flow diagram of a process in accordance with the invention; and

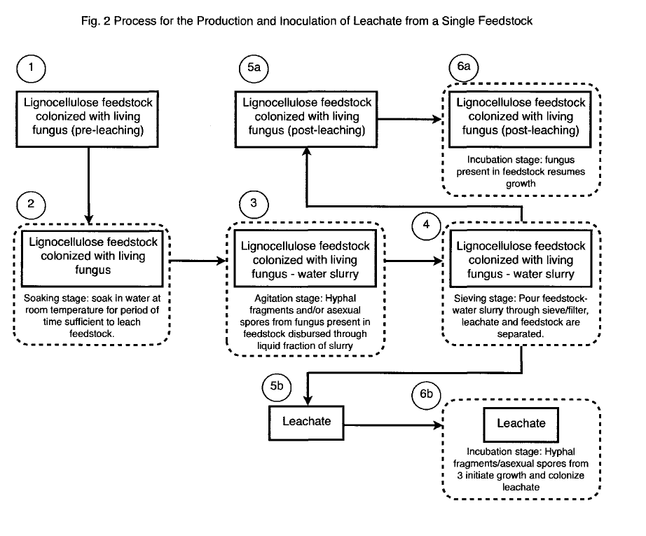

FIG. 2 illustrates a flow diagram of a modified process in accordance with the invention.

Referring to FIG. 1, in step 1 of the process, a feedstock of a lignocellulose substrate 10 is obtained and colonized with a given species of fungi. This substrate can encompass a spent mushroom substrate from the mushroom cultivation industry, dehydrated or non-dehydrated substrate, pre or post sporocarp development.

In step 2, the colonized feedstock is added to water (or water is added to the feedstock) within a container to form a liquid medium.

The substrate is soaked in the water at room temperature for a period sufficient to leach water soluble compounds from the feedstock and form a liquid medium, i.e. a feedstock-water slurry. The water soluble compounds have been found to include (1) sugar alcohol, such as glycerin, (2) a phenolic compound, such as 2,6 Dimethoxy phenol and (3) a fatty acid, such as pentadecanoic acid. The compounds may also include at least one of a carbonyl and a phenyl wherein the carbonyl is one of heptanal and pentanal and the phenyl is benzoic acid.

Thereafter, in step 3, the feedstock-water slurry is poured through a sieve/filter to separate the solids from the liquid leachate.

In step 4, the liquid leachate is removed and in step 5 is subjected to heat for pasteurization or sterilization particularly where a specific application of the leachate requires such.

Optionally, the feedstock substrate may be soaked in water heated to a temperature of at least 180° F. In this case, the obtained leachate need not be subjected to heat for pasteurization or sterilization.

Also, as an option, the solid substrate need not be removed from the liquid leachate.

Referring to FIG. 2, in step 1 of a process for the production and inoculation of leachate from a single feedstock, a feedstock of a lignocellulose substrate is obtained and colonized with a living and viable fungus which is not dehydrated and can be pre or post sporocarp development (pre-leaching).

As above, in step 2, water is added to the colonized feedstock in a container and the feedstock soaked at room temperature for a period sufficient to leach water soluble compounds from the feedstock and to form a feedstock-water slurry.

In step 3, the slurry is then agitated vigorously to release and distribute fragments of mycelium and/or asexual spores from the feedstock into the water/leachate, for example, in experimental trials, a flask containing the slurry was vigorously shaken for this purpose.

In step 4, the slurry is poured through a sieve/filter to separate the leachate from the solids.

Next, in step 5a, the solid feedstock with living fungus is removed from the liquid leachate and, in step 6a, the feedstock is then incubated (i.e. post-leaching) to achieve a desired colonization. In this regard, the fungus is allowed to continue growing in the feedstock in order to bind loose particles of substrate into a solid mass.

In step 5b, the leachate is removed and, in step 6b, is then incubated to allow the fungus to continue growing. The fragments of mycelium/asexual spores present in the leachate (provided by the fungus in the feedstock via agitation) will initiate growth, eventually forming a solid sheet of mycelium on the surface of the leachate and/or growing through the volume of the leachate, if gelatinized, to obtain 3D colonization.

In another embodiment, a leachate may be specifically derived from rye straw colonized and decomposed by a primary saprophyte. This leachate medium shares the same general functionality of other leachate media described here, but has a unique stimulatory effect on the germination of chlamydospores (an asexual spore produced in the mycelium of many fungi).

Chlamydospores in the presence of this specific leachate medium germinate at percentages of over 90% by 24 hours after inoculation, as compared to much lower germination rates in other traditional and leachate media. The specific components of this leachate, determined through mass spectrometry, are listed below:

Butanoic acid 3 methyl
butyrolactone
cyclohexanone
glycerin
2-propanamine
2 5 dimethyl 4 hydroxy 3(2H) furanone
Formic acid ethenyl ester
maltol
pentanoic acid 4 oxo
4H pyran 4 one 2 3 dihydro 3 5 dihydroxy 6 methyl
isosorbide
propanedioic acid phenyl
isosorbide
4H 1 3 oxazine 5 6 dihydro 2 4 4 tetramethyl
Phenol 2 6 dimethoxy
d mannitol 1 4 anhydro
D erythro penose 2 deoxy
guanosine
heptanamine 5 methyl
heptane 3 methylene

### Applications

### 1) Culture Medium

Fungi can be cultivated on leachate as a stand-alone medium, or gelatinized using agar agar for use as tissue suspension media or plate culture media.
Conventional culture media require many chemical ingredients, which utilized within large scale processes may be prohibitively costly. Because there is no need for additional chemical ingredients, utilization of leachate in place of conventional culture media may lead to reduced cost at scale.
Using a fungus as the primary processing agent in creating leachate culture media eliminates the need for complex chemical composition, and the wide range of lignocellulose sources which can be converted via fungus to leachate culture media eliminates dependence on economically important food crops and elaborate manufacturing processes.
Colonized fungal leachate can be utilized as an inoculum for other substrates
The simplicity of the production process, the availability of low cost lignocellulose sources, the availability of spent mushroom substrate waste streams, and the ability to utilize fungally colonized lignocellulose substrate that is dehydrated, non-dehydrated, pre and post sporocarp production, make fungal leachate a malleable and scalable medium for processes requiring the mass cultivation of fungal biomass.
   2) As an Additive to Solid Substrate Prior to Inoculation with Fungi
Leachate can be added to lignocellulose substrate prior to inoculation of fungi as an additional nutrient source and growth stimulant.
Many species of fungi depend on nutrients made available by earlier decomposition of a given substrate as a requirement for vegetative growth and/or sporocarp formation. The addition of leachate to virgin lignocellulose substrate may be an accessible method of providing these nutrients for the cultivation of these species.
   3) Leachate Extracted from, and Inoculated by, a Single Fungally Colonized Lignocellulose Feedstock
Typically fungal cultivation methods require a substrate or culture medium be prepared through one process, and the inoculum (fungus grown on a carrier substrate) prepared through another process, then inoculum is introduced to the substrate (inoculated). For example, a liquid culture medium would typically be inoculated with a plate tissue culture. The ability of a single mass of fungally colonized lignocellulose to provide both the growth medium and inoculum in a single procedure greatly simplifies this process.
The process may be a simple and inexpensive method at scale of transferring a given genetic individual of fungus from solid substrate, to liquid (leachate) culture, and back to solid substrate.

The following are examples of the method of the invention.

### EXAMPLE 1

### Production of Leachate

### Option 1

1) Add 1 liter of dehydrated 50/50 wheat straw-cotton seed hull substrate previously colonized with Pleurotus ostreatus to a container, breaking apart substrate into particles
2) Add 1 liter of room temperature water to the container with substrate
3) Agitate slurry and/or place cover over container in order to fully submerge substrate in water
4) Soak substrate-water slurry for a period of 4 to 18 hours
5) Pour slurry through sieve to separate solids from liquid leachate, pressing solids in order to reclaim as much leachate as possible
6) Filter or heat sterilize leachate
7) Further process and inoculate leachate according to desired application.

### Option 2

1) Add 1 liter of dehydrated 50/50 wheat straw-cotton seed hull substrate previously Colonized with Pleurotus ostreatus to a container, breaking apart substrate into particles
2) Add 3 liters of room temperature water to the container with substrate
3) Agitate slurry and/or place cover over container in order to fully submerge substrate in water
4) A) Soak substrate-water slurry for a period of 4 to 18 hours or B) continue to step 5
5) Heat sterilize leachate by pressure cooking at 15 psi for 1 hour
6) A) Pour slurry through sieve to separate solids from liquid leachate, pressing solids in Order to reclaim as much leachate as possible, or B) Do not separate solids from leachate and continue to step 7
7) Further process and inoculate leachate according to desired application

### EXAMPLE 2

### Cultivating Sheets of Mycelium on Leachate

1) Produce leachate as described in example 1
2) Fill 1 liter vessel with leachate
3) Process in pressure sterilizer at 15 psi for 1 hour
4) Pour 200 ml leachate into a blender, add 1 culture plate as inoculum to the blender and blend to distribute inoculum
5) Pour inoculated leachate from blender back into 1 liter vessel
6) Add 2 ml of 3.5% hydrogen peroxide to vessel, agitate, and loosen lid of vessel to allow for respiration
7) Incubate to cultivate desired mycelial sheet

### EXAMPLE 3

### Leachate Tissue Suspension Media

1) Produce leachate as described in example 1
2) Add 2 g agar agar per liter of leachate
3) Process in pressure sterilizer at 15 psi for 1 hour
4) Let media cool while stirring
5) Inoculate according to standard practices
6) Incubate until desired colonization is achieved

### EXAMPLE 4

### Gelatinized Leachate for Culture Plates

1) Produce leachate as described in example 1
2) Add 15 g agar agar per liter of leachate
3) Process in pressure sterilizer at 15 psi for 1 hour
4) Pour appropriate amount of liquid into petri dishes
5) Let cool to set

### EXAMPLE 5

### Addition of Leachate to Solid Substrate

1) Produce leachate as described in example 1
2) Prepare dry components of substrate
3) A) Use leachate as sole moisture component of substrate
   B) Use leachate as a fraction of moisture component of substrate
4) Pasteurize or sterilize substrate according to common practices
5) Inoculate and incubate substrate according to common practices

### EXAMPLE 6

### Production and Inoculation of Leachate from Single Mass of Fungally Colonized Lignocellulose

1) Prepare 5 liters of wheat straw substrate and inoculate with Pleurotus ostreatus per standard cultivation techniques
2) Incubate until substrate is fully colonized by fungus
3) Remove colonized substrate from bag, break apart into smaller pieces, and place in container
4) Add 3 liters of sterile deionized water to container with substrate, ensuring that substrate is fully submerged in water
5) Allow substrate to soak for at least 7 hours
6) Drain liquid from substrate by pouring through filter or sieve
7) Press substrate in tincture press sprayed with 70% isopropanol in order to reclaim as much leachate as possible and remove excess moisture from substrate
8) Pour leachate into 1 liter bottles
9) Place substrate back in bag
10) Incubate substrate until desired regrowth and/or fruit body production has been achieved
11) Incubate leachate until cohesive sheet of P. ostreatus mycelium is present on the top surface of the fluid

### Experimental Results

Over the course of experimentation, mass spectrometry was performed on leachate samples derived from feedstocks including 5 different species of basidiomycetes grown on multiple substrates, both before and after heat sterilization. The exact components are variable depending on the substrate, species, and whether or not it has been heat processed, but the following general components are constant:
A) Sugar alcohols: most commonly glycerol, but many sugar alcohols present
B) Phenolics: most commonly 2,6 Dimethoxy phenol, but may also contain vanillin and/or vanillic acid
C) Fatty acids: most commonly pentadecanoic acid

Most, but not all varieties of leachate also contain the following (depending on substrate and species composing the feedstock)
D) Carbonyls, such as heptanal and pentanal
E) Phenyls, such as benzoic acid

Below is an example of mass spectrometry results for a leachate sample derived from feedstock composed of hemp substrate colonized by a higher basidiomycete, both pre and post-heat sterilization in a pressure sterilizer at 15 psi for 1 hour. The substrate was dehydrated prior to sporocarp development, and leachate was produced.

Sample mass spectrometry result, leachate sample pre-heat sterilization

| rt | intensity | probable ID |
|---|---|---|
| 4.765E+00 | 5.810E+06 | tetrahydro 4H pyran 4 ol |
| 4.784E+00 | 6.500E+06 | hexylene glycol |
| 5.960E+00 | 9.614E+06 | 5 hexen 2 one |
| 6.090E+00 | 7.21E+06 | 2 5-dimethyl-4-hydroxy-3(2H)-furanone |
| 6.558E+00 | 5.887E+06 | maltol |
| 6.800E+00 | 6.35E+06 | 4H pyran 4 one 2 3 dihydro 3 5 dihydroxy |
| | | 6 methyl |
| 7.001E+00 | 6.664E+06 | benzoic acid |
| 8.056E+00 | 6.28E+06 | phlorogllucitol |
| 8.254E+00 | 8.45E+06 | D-erythro pentose 3 deoxy |
| 8.343E+00 | 1.167E+07 | phenol 2 6 dimethoxy |
| 8.691E+00 | 6.508E+06 | vanilin |
| 8.778E+00 | 1.093E+07 | 2 pentene 3 methyl [Z] |
| 9.769E+00 | 1.38E+07 | nicoNnyl alcohol |
| 9.822E+00 | 1.177E+07 | benzoic acid 4 hydroxy 3 methoxy methyl |
| | | ester |
| 1.022E+01 | 1.376E+07 | xylitol |
| 1.113E+01 | 1.162E+07 | ethanone 1-(4 hydroxy 3 5 dimethoxyphenyl |
| 1.171E+01 | 1.437E+07 | pentadecanoic acid |
| 1.270E+01 | 9.858E+06 | octadecanoic acid |
| 1.381E+01 | 7.688E+06 | dicyclohexyl methyl phosphonate |
| 1.441E+01 | 8.000E+07 | benzene 3 butenyl |
| 1.502E+01 | 9.612E+06 | hexadecane |
| 1.572E+01 | 7.047E+06 | 4 octen 3 one |
| 1.596E+01 | 7.431E+06 | 1 octanol 2 butyl |
| 1.617E+01 | 1.006E+07 | 2 methyl octadecane |
| 1.625E+01 | 7.998E+06 | benzene 1 1′(1,3,5 hexatriene 1,6 diy)bis--- |
| 1.687E+01 | 1.007E+07 | 1-octanol 2 butyl |
| 1.713E+01 | 1.988E+07 | 1,1′,3′,1″ terphenyl 5′ phenyl |
| 1.734E+01 | 8.292E+06 | eicosane |
| 1.774E+01 | 7.361E+06 | thiphene |
| 1.791E+01 | 7.700E+06 | hexadecane |
| 1.830E+01 | 1.464E+07 | norethnodrel |

Sample mass spectrometry result, leachate sample post-heat sterilization

| rt | intensity | probable ID |
|---|---|---|
| 4.75E+00 | 5.855E+06 | oxepanone |
| 4.771E+00 | 1.228E+07 | hexylene glycol |
| 4.844E+00 | 4.819E+06 | cyclohexanone |
| 5.560E+00 | 6.597E+06 | D erythro pentose 2 deoxy |
| 5.710E+00 | 5.95E+06 | glycerin |
| 5.750E+00 | 6.163E+06 | glycerin |
| 5.880E+00 | 6.68E+06 | 3 penten 2 ol |
| 5.981E+00 | 1.06E+07 | propanoic acid 2-oxo ethyl ester |
| 6.087E+00 | 9.113E+06 | 2,5 dimethyl 4 hydroxy 3(2H) furanone |
| 6.075E+00 | 5.816E+06 | 1---propanamine N,2 dimethyl---N nitroso |
| 6.576E+00 | 6.510E+06 | maltol |
| 6.825E+00 | 5.95E+06 | 4H pyran 4 one 2,3 dihydro 3 5 dihydroxy |
| | | 6 methyl |
| 6.957E+00 | 5.252E+06 | cycloserine D |
| 7.000E+00 | 6.293E+06 | benzoic acid |
| 7.350E+00 | 1.889E+07 | D-glucitol |
| 7.630E+00 | 1.219E+07 | D-erythro pentose 2 deoxy |
| 8.021E+00 | 5.843E+06 | 2 heptanal 5 ethyl |
| 8.064E+00 | 8.233E+06 | 2 heptanol 5 ethyl |
| 8.150E+00 | 7.011E+06 | 2 nonanol |
| 8.325E+00 | 1.603E+07 | phenol 2,6 dimethoxy |
| 8.475E+00 | 8.373E+06 | 1,3 butanediol |
| 8.633E+00 | 5.535E+06 | acetic acid 2 ethyl hexyl ester |
| 8.699E+00 | 5.884E+06 | guanosine |
| 8.790E+00 | 7.980E+06 | 1 pentene 2,3 dimethyl |
| 8.924E+00 | 6.234E+06 | 2 propanone 1-(1,3-dioxolan-2-yl) |
| 9.798E+00 | 5.972E+06 | vanilic acid |
| 9.832E+00 | 9.508E+06 | vanilic acid methyl ester |
| 1.065E+01 | 5.841E+06 | benzeneacetic acid 4 hydroxy 3 methoxy |
| 1.113E+01 | 7.475E+06 | acetosyringone |
| 1.171E+01 | 6.861E+06 | pentadecanoic acid |

Below are other compounds frequently found in common between leachates produced from different feedstocks:
4H pyran 4 one 2 3 dihydro 3 5 dihydroxy—A plant phytochemical, present in all leachate samples both pre and post-heat sterilization. This is also thought to be a degradation product of some hexose. This is likely being derived from the substrate and not a specific result of fungal decomposition of the substrate.
Maltol—Used as an odor and flavor enhancer. This is present in samples from all species tested; found in both pre and post-heat sterilized samples in two species, in post-heat sterilized only in two species, and pre-heat sterilized sample for one species.
Isosorbide—May have anti-microbial properties. This is present in post-heat sterilized samples only of three out of five species tested. The lack of isosorbide in pre-heat sterilized samples indicates this may be a product of heat sterilization.
VanillinNanillic acid—By-products of lignin decomposition, these are products of fungal decomposition of the substrate. These were found in samples from 4 out of 5 species, both pre and post-heat sterilization.

Many of these components, especially sugar alcohols, phenolics, and phenyls, are known to be by-products of fungal lignin decomposition. It has been demonstrated that leachate derived from virgin substrate (not colonized by a fungus) does not function as a fungal culture medium. Therefore, the fungal decomposition of the lignocellulose substrate prior to soaking to produce leachate is necessary to produce the components outlined above that are responsible for the functionality of the leachate.

The invention thus provides a process that employs leachate as a liquid culture medium for cultivating fungi and, particularly, a fungal leachate that does not require additional nutrient components.

The invention further provides a process for producing leachate in a less energy intensive manner than prior art processes.

What is claimed is:

1. A fungal leachate solution containing a water soluble fraction of fungally colonized and decomposed lignocellulosic substrate.

2. A method of producing a fungal leachate solution comprising the steps of
obtaining a feedstock of lignocellulosic substrate;
colonizing said substrate with a selected fungi; and
adding water to said colonized substrate to form a liquid medium containing at least one of sugar alcohol, a phenolic compound and a fatty acid.

3. A method as set forth in claim 2 wherein said sugar alcohol is glycerin, said phenolic compound is 2,6 Dimethoxy phenol and said fatty acid is pentadecanoic acid.

4. A method as set forth in claim 2 wherein said liquid medium contains at least one of a carbonyl and a phenyl.

5. A method as set forth in claim 4 wherein said Carbonyl is one of heptanal and pentanal and said phenyl is benzoic acid.

6. A method as set forth in claim 2 wherein said feedstock is soaked in said water at room temperature for a period sufficient to leach water soluble compounds and which further comprises the step of thereafter removing solid substrate while retaining liquid leachate.

7. A method as set forth in claim 2 wherein said feedstock is soaked in said water and heated to a temperature of at least 180° Fahrenheit for a period sufficient to leach water soluble compounds and which further comprises the step of thereafter removing solid substrate while retaining liquid leachate.

8. A method as set forth in claim 2 wherein said feedstock is soaked in said water for a period sufficient to leach water soluble compounds and to form a slurry comprised of liquid leachate and solid substrate.

9. A method as set forth in claim 2 wherein said feedstock is dehydrated prior to said step of adding water.

10. A method as set forth in claim 2 wherein said feedstock of lignocellulosic substrate is comprised of spent mushroom substrate characterized in having had at least one flush of mature sporocarps harvested therefrom.

11. A method as set forth in claim 2 wherein said feedstock of lignocellulosic substrate is comprised of spent mushroom substrate characterized in not having had a flush of mature sporocarps harvested therefrom.

12. A method comprising the steps of
obtaining a feedstock of lignocellulosic substrate;
colonizing said substrate with a selected fungi;
adding water to said colonized substrate to form a liquid medium containing at least one of sugar alcohol, a phenolic compound and a fatty acid;
inoculating said liquid medium with a selected fungus; and
thereafter incubating said inoculated liquid medium to effect metabolizing and colonization of said liquid medium.

13. A method comprising the steps of
obtaining a feedstock of lignocellulosic substrate;
colonizing said substrate with a selected fungi;
adding water to said colonized substrate to form a liquid medium containing at least one of sugar alcohol, a phenolic compound and a fatty acid;
adding said liquid medium to a solid lignocellulosic substrate;
thereafter inoculating said solid lignocellulosic substrate with a selected fungus; and
thereafter incubating said inoculated lignocellulosic substrate to effect metabolizing and colonization of said lignocellulosic substrate.

14. A fungal leachate solution containing a water soluble fraction of fungally colonized and decomposed rye straw.

15. A method of producing a fungal leachate solution comprising the steps of
obtaining a feedstock of rye straw;
colonizing and decomposing said feedstock with a primary saprophyte; and
adding water to said colonized substrate to form a liquid medium.
